(19) United States
(12) Patent Application Publication
(10) Pub. No.: US 2004/0133179 A1
(43) Pub. Date: Jul. 8, 2004

(54) Thin, flexible micro-hook system for a feminine care article

(75) Inventors: Paul S. Steger, Neenah, WI (US); Christina M. Rhein, Neenah, WI (US); David R. Steinike, Appleton, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/336,136

(22) Filed: Jan. 3, 2003

(51) Int. Cl. A61F 13/15; A61F 13/20

(52) U.S. Cl. 604/385.04

(57) ABSTRACT

An absorbent feminine care article (20) having a longitudinal direction (22), a lateral direction (24), first and second longitudinally opposed end portions (72, 72a), and a intermediate portion (76) located between the end portions. The article (20) comprises an absorbent body (30) sandwiched between a cover (26) and a baffle (28). A first side-panel (42) is attached to a first, lateral side region (60) of the intermediate portion (76) of the article, and the first side-panel (42) includes a first panel-fastener component (44) operatively joined to an engagement surface (50) of the first side-panel. A second side-panel (42a) is attached to a second, lateral side region (60a) of the intermediate portion (76) of the article, and the second side-panel (42a) includes a second panel-fastener component (44a) operatively joined to an engagement surface of the second side-panel. In a particular feature, the first and second side panels can have a selected overlap distance (58), as observed when the side panels (42, 42a) are arranged in their storage position. In other features, the article can have a selected panel-edge distance (66) and/or a selected, outboard fastener-edge distance (68).

### FIELD OF THE INVENTION

The present invention relates to a fastening system for an absorbent article. More particularly, the present invention pertains to a flexible, interengaging, mechanical fastening system for a feminine care article, such as a feminine care pad.

### BACKGROUND OF THE INVENTION

Absorbent products intended to absorb discharged body fluids are well known in the art. Such absorbent products generally comprise a fibrous mass or other absorbent body which can absorb and hold the body fluids. Similarly, it is well known that, feminine care articles have been employed to absorb and hold liquids, such as urine and/or menses. In particular arrangements, the feminine care articles have included wing portions which can help to hold the article in place at a selected location in a wearer's undergarment. Various fasteners have been employed to secure the wing portions in a desired configuration during ordinary use. The fasteners have included adhesive fasteners as well as mechanical fasteners, and the mechanical fasteners have included conventional, hook-and loop fasteners.

Conventional fasteners, however, have not provided desired combinations of securement and comfort, particularly when the wearer is moving and highly active. When such conventional fasteners have been constructed to provide desired securement, the articles have been excessively uncomfortable during active wear. When such conventional fasteners have been constructed to provide desired levels of comfort, the level of securement has been poor. As a result, there has been a continued need for improved fastening systems that provide more secure levels of fastening along with increased levels of comfort.

### BRIEF DESCRIPTION OF THE INVENTION

Generally stated, the present invention provides an absorbent feminine care article having a longitudinal direction, a lateral direction, first and second longitudinally opposed end portions, and a intermediate portion located between the end portions. The article comprises an absorbent body sandwiched between a cover and a baffle. A first side-panel is attached to a first, lateral side region of the intermediate portion of the article, engagement surface of the first side-panel. A second side-panel is attached to a second, lateral side region of the intermediate portion of the article, and the second side-panel includes a second panel-fastener component operatively joined to an engagement surface of the second side-panel. In a particular feature, the first and second side panels can have a selected overlap distance, as observed when the side panels are arranged in their storage position. In other features, the article can have a selected panel-edge distance and/or a selected fastener-edge distance.

By incorporating its various features and configurations, the article of the invention can be less susceptible to premature pop-opens, and can provide less irritation and greater comfort to the wearer. Additionally, the article can be less susceptible to premature leakage, and can provide greater confidence to the wearer.

### BRIEF DESCRIPTION OF THE DRAWINGS

The various features, aspects and advantages of the present invention will become better understood with reference to the following description, appended claims and accompanying drawings where:

FIG. 1 shows a plan view of a garment-side of a representative feminine care article in which the side-panel portions are generally arranged in a storage position.

FIG. 1A shows a schematic view of a representative lateral, transverse cross-section through the article illustrated in FIG. 1.

FIG. 2 shows a partially cut-away, top, plan view of a bodyside of a representative feminine care article in which side-panel portions are arranged in a laterally-extended position.

FIG. 2A shows a schematic view of a representative, transverse cross-section through the article illustrated in FIG. 2.

FIG. 3 representatively shows a plan view of an appointed loop-surface of a side-panel portion, which during use, can face outwardly, away from the wearer's body.

FIG. 3A representatively shows a plan view of an appointed hook-surface of a side-panel component, which during use, can face inwardly, towards the wearer's body.

FIG. 4 representatively shows a representative bottom-side view in which the side-panel portions of the article are wrapped about a wearer's undergarment, and operatively engaged generally adjacent an outwardly-facing surface of the undergarment.

### DETAILED DESCRIPTION OF THE INVENTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

By the terms “particle,” “particles,” “particulate,” “particulates” and the like, it is meant that the adsorbent material is generally in the form of discrete units. The units can comprise granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

As used herein, the term “nonwoven” refers to a fabric web that has a structure of individual fibers or filaments which are interlaid, but not in an identifiable repeating manner.

As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

As used herein, the phrase “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

“Coform” as used herein is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

As used herein, the phrase “complex liquid” describes a liquid generally characterized as being a viscoelastic liquid comprising multiple components having inhomogeneous physical and/or chemical properties. It is the inhomogeneous properties of the multiple components that challenge the efficacy of an adsorbent material in the handling of complex liquids. In contrast with complex liquids, simple liquids, such as, for example, urine, physiological saline, water and the like, are generally characterized as being relatively low-viscosity and comprising one or more components having homogeneous physical and/or chemical properties. As a result of having homogeneous properties, the one or more components of simple liquids behave substantially similarly during absorption or adsorption.

Although a complex liquid is generally characterized herein as including specific components having inhomogeneous properties, each specific component of a complex liquid generally has homogeneous properties. Consider for example a representative complex body-liquid having three specific components: red blood cells, blood protein molecules and water molecules. Upon examination, one skilled in the art could easily distinguish between each of the three specific components according to their generally inhomogeneous properties. Moreover, when examining a particular specific component such as the red blood cell component, one skilled in the art could easily recognize the generally homogeneous properties of the red blood cells.

As used herein, the phrase “absorbent article” refers to devices which absorb and contain body liquids, and more specifically, refers to devices which are placed against or near the skin to absorb and contain the various liquids discharged from the body. The term “disposable” is used herein to describe absorbent articles that are not intended to be laundered or otherwise restored or reused as an absorbent article after a single use. Examples of such disposable absorbent articles include, but are not limited to: health care related products including surgical drapes, gowns, and sterile wraps; personal care absorbent products such as feminine hygiene products (e.g., sanitary napkins, pantiliners, tampons, interlabial devices and the like), infant diapers, children's training pants, adult incontinence products and the like; as well as absorbent wipes and covering mats.

Disposable absorbent articles such as, for example, many of the feminine care absorbent products, can include a liquid pervious topsheet, a substantially liquid impervious backsheet joined to the topsheet, and an absorbent core positioned and held between the topsheet and the backsheet. The topsheet is operatively permeable to the liquids that are intended to be held or stored by the absorbent article, and the backsheet may be substantially impermeable or otherwise operatively impermeable to the intended liquids. The absorbent article may also include other components, such as liquid wicking layers, liquid intake layers, liquid distribution layers, transfer layers, barrier layers, and the like, as well as combinations thereof. Disposable absorbent articles and the components thereof, can operate to provide a body-facing surface and a garment-facing surface. As used herein, “body-facing surface” means that surface of the article or component which is intended to be disposed toward or placed adjacent to the body of the wearer during ordinary use, while the “outward surface” or “outward-facing surface” is on the opposite side, and is intended to be disposed to face away from the wearer's body during ordinary use. The outward surface may be arranged to face toward or placed adjacent to the wearer's undergarments when the absorbent article is worn.

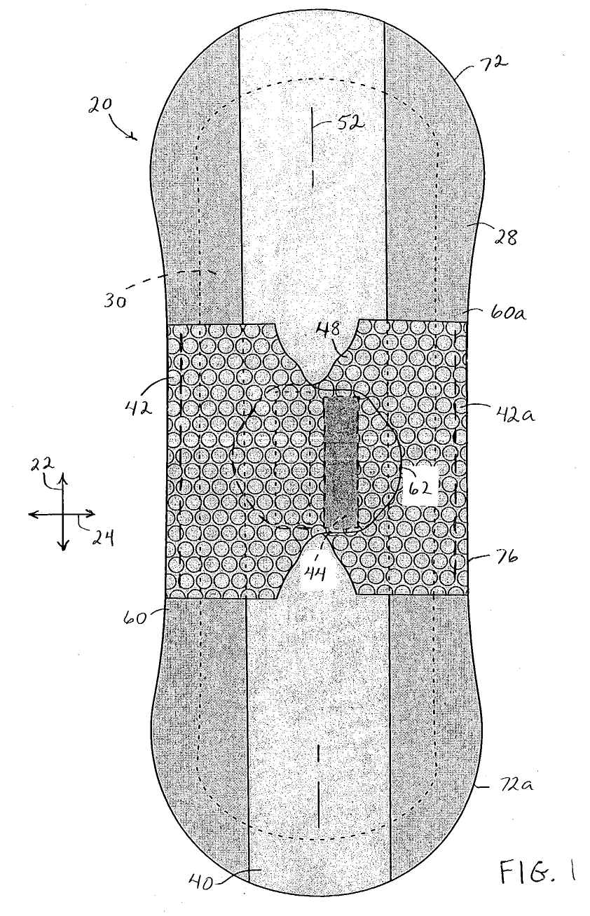

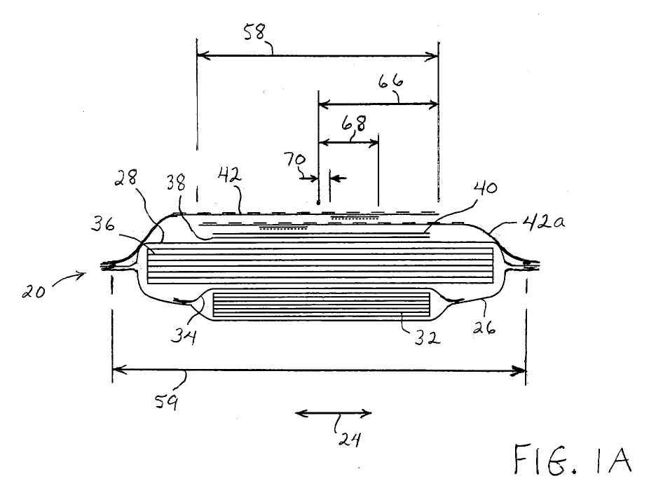

FIGS. 1 and 1A, illustrate an example of a suitable article, such as the representatively shown feminine care article, which is configured to incorporate the present invention. The feminine care article can, for example, be a feminine care pad or napkin 20, and the article can have a lengthwise longitudinal direction 22, a transverse, laterally extending, cross-direction 24, first and second longitudinally opposed end portions 72 and 72a, and a intermediate portion 76 located between the end portions. As representatively shown, the longitudinal dimension of the article is relatively larger than the lateral dimension of the article. The article 20 can include a topsheet or cover 26, a baffle 28, and an absorbent structure 30 positioned between the cover and baffle. A first side-panel 42 can be attached to a first, lateral side region 60 of the intermediate portion 76 of the article, and the first side-panel 42 can include a first panel-fastener component 44 which is operatively joined to an engagement surface 50 of the first side-panel. A second side-panel 42a can be attached to a second, lateral side region 60a of the intermediate portion 76 of the article, and the second side-panel 42a can include a second panel-fastener component 44a which is operatively joined to an engagement surface of the second side-panel. In a particular feature, the first and second side panels can have a selected overlap distance 58 (e.g. FIG. 1A), as observed when the side panels 42 and 42a are arranged in their storage position. In other features, the article can have a selected panel-edge distance 66 and/or a selected fastener-edge distance 68, as measured from a longitudinally-extending centerline 52 (e.g. FIG. 1) of the article. A desired configuration can, for example, include an overlap distance of at least a minimum of about 40 mm when the side-panels are arranged in their storage position.

By incorporating its various aspects, features and configurations (alone or in desired combinations), the article of the invention can be less susceptible to premature pop-opens, and can provide less irritation and greater comfort to the wearer. Additionally, the article can be less susceptible to premature leakage, and can provide greater confidence to the wearer.

The cover 26 may include a layer constructed of any operative material, and may be a composite material. For example, the cover layer can include a woven fabric, a nonwoven fabric, a polymer film, a film-fabric laminate or the like, as well as combinations thereof. Examples of a nonwoven fabric include, spunbond fabric, meltblown fabric, coform fabric, a carded web, a bonded-carded-web, a bicomponent spunbond fabric or the like as well as combinations thereof. For example, the cover layer can include a woven fabric, a nonwoven fabric, a polymeric film that has been configured to be operatively liquid-permeable, or the like, as well as combinations thereof. Other examples of suitable materials for constructing the cover layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable cover layer material can include a bonded-carded-web composed of polypropylene and polyethylene, such as has been used as a cover stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. In a desired arrangement, the cover layer 26 can be configured to be operatively liquid-permeable with regard to the liquids that the article is intended to absorb or otherwise handle. The operative liquid-permeability may, for example be provided by a plurality of pores, perforations, apertures or other openings, as well as combinations thereof, that are present or formed in the cover layer. The apertures or other openings can help increase the rate at which bodily liquids can move through the thickness of the cover layer and penetrate into the other components of the article (e.g. into the absorbent structure 30). The selected arrangement of liquid-permeability is desirably present at least on an operative portion of the cover layer that is appointed for placement on the body-side of the article. The cover layer 26 can provide comfort and conformability, and can function to direct bodily exudates away from the body and toward the absorbent structure 30. In a desired feature, the cover layer 26 can be configured to retain little or no liquid in its structure, and can be configured to provide a relatively comfortable and non-irritating surface next to the body-tissues of a female wearer. The cover layer 26 can be constructed of any material which is also easily penetrated by bodily fluids that contact the surface of the cover layer.

The cover 26 can also have at least a portion of its bodyside surface treated with a surfactant to render the cover more hydrophilic. The surfactant can permit arriving bodily liquids to more readily penetrate the cover layer. The surfactant may also diminish the likelihood that the arriving bodily fluids, such as menstrual fluid, will flow off the cover layer rather than penetrate through the cover layer into other components of the article (e.g. into the absorbent body structure). In a particular configuration, the surfactant can be substantially evenly distributed across at least a portion of the upper, bodyside surface of the cover 26 that overlays the upper, bodyside surface of the absorbent.

The cover 26 may be maintained in secured relation with the absorbent structure 30 by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The cover 26 typically extends over the upper, bodyside surface of the absorbent structure, but can alternatively extend around the article to partially or entirely, surround or enclose the absorbent structure. Alternatively, the cover 26 and the baffle 28 can have peripheral margins which extend outwardly beyond the terminal, peripheral edges of the absorbent structure 30, and the extending margins can be joined together to partially or entirely, surround or enclose the absorbent structure.

The baffle 28 may include a layer constructed of any operative material, and may or may not be configured to be liquid-permeable. In a particular configuration, the backsheet or baffle 28 may be configured to provide an operatively liquid-impermeable layer. The baffle may, for example, include a polymeric film, a woven fabric, a nonwoven fabric or the like, as well as combinations or composites thereof. For example, the baffle may include a polymer film laminated to a woven or nonwoven fabric. In a particular feature, the polymer film can be composed of polyethylene, polypropylene, polyester or the like, as well as combinations thereof. Additionally, the polymer film may be micro-embossed. Desirably, the baffle 28 can operatively permit a sufficient passage of air and moisture vapor out of the article, particularly out of an absorbent (e.g. storage or absorbent structure 30) while blocking the passage of bodily liquids. An example of a suitable baffle material can include a breathable, microporous film, such as a HANJIN Breathable Baffle available from Hanjin Printing, Hanjin P&C Company Limited, a business having offices located in Sahvon-li.Jungan-mvu.Kongiu-City, Chung cheong nam-do, Republic of South Korea. The baffle material is a breathable film, which is white in color, dimple embossed., and contains: 47.78% calcium carbonate, 2.22% TiO₂, and 50% polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable baffle material can include a closed cell polyolefin foam. For example, a closed cell polyethylene foam may be employed. Still another example of a baffle material would be a material that is similar to a polyethylene film which is used on commercially sold KOTEX brand pantiliners, and is obtainable from Pliant Corporation, a business having offices located in Schaumburg, Illinois, USA.

The structure of the absorbent body 30 can be operatively configured to provide a desired level of absorbency or storage capacity. More particularly, the absorbent body can be configured to hold a liquid, such as urine, menses, other complex liquid or the like, as well as combinations thereof. As representatively shown, the absorbent body can include a matrix of absorbent fibers and/or absorbent particulate material, and the absorbent fiber can include natural and/or synthetic fiber.

The absorbent structure 30 may also include superabsorbent material. Superabsorbent materials suitable for use in the present invention are known to those skilled in the art, and may be in any operative form, such as particulate form. Generally stated, the water-swellable, generally water-insoluble, hydrogel-forming polymeric absorbent material (superabsorbent) is capable of absorbing at least about 10, desirably about 20, and possibly about 100 times or more its weight in water. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company, Hoechst Celanese Corporation, Allied Colloid Inc., and Stockhausen, Inc.

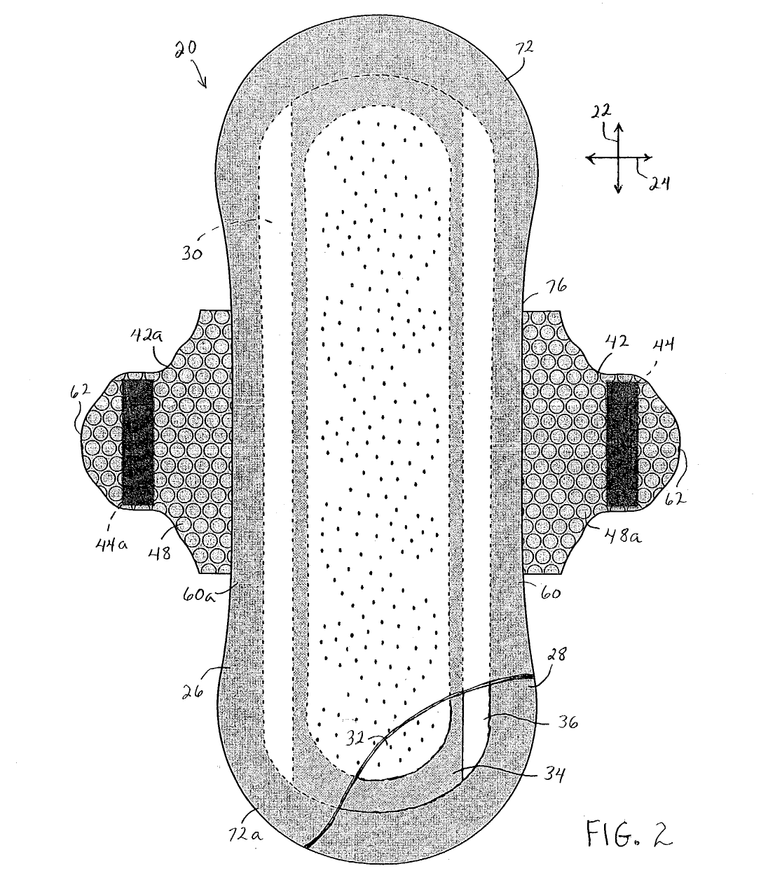

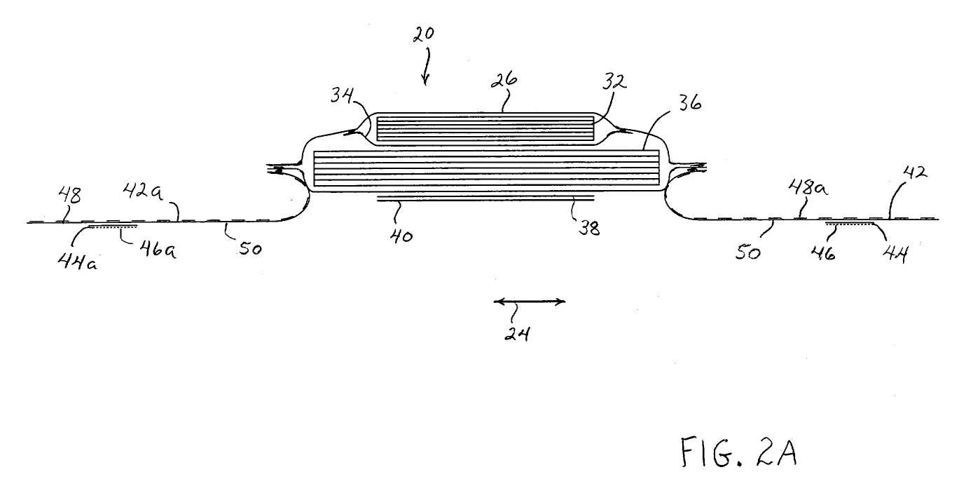

As representatively shown, the absorbent body 30 can comprise a composite structure having a selected plurality of strata or layers. With reference to FIGS. 1 through 2A, the absorbent composite can, for example, include an intake layer 32, a distribution or transfer layer 34, and/or an absorbent shaping layer 36, as well as any operative combination thereof. As representatively shown, the absorbent body can include an absorbent pad, shaping layer 36 which is positioned between the cover 26 and the baffle 28. Additionally, the absorbent body can include an intake layer 32 which is positioned between cover 26 and pad shaping layer 36. The absorbent body can further include a transfer layer 34 which is positioned between the intake layer 32 and the pad shaping layer 36.

The intake layer 32 can provide a desired intake of liquid and distribution of the liquid. The intake layer may include natural fibers (e.g. cellulose fibers), synthetic fibers, superabsorbent materials, a woven fabric; a nonwoven fabric; a wet-laid fibrous web; a substantially unbonded airlaid fibrous web; an operatively bonded, stabilized-airlaid fibrous web; a multifunctional stabilized-airlaid fibrous web; or the like, as well as combinations thereof.

In a particular arrangement, the intake layer can be a thermally-bonded, stabilized-airlaid fibrous web having a basis weight of about 175-200 g/m², and a density of about 0.06-0.08 grams/cm³. Suitable stabilized-airlaid webs are available from Concert Fabrication, a business having offices located in Gatineaux, Quebec, Canada.

The transfer layer 34 can provide the function(s) of transfer delay, liquid and spreading. In a particular arrangement, the transfer layer 34 may be configured to provide a selected delay in the transfer or other distribution of the incoming liquid into the shaping layer 36.

The transfer layer 34 may include a wettable spunbonded, nonwoven fabric, a through-air-bonded-carded web or the like, as well as combinations thereof. In a particular arrangement, the transfer layer can be a spunbond fabric having a basis weight of about 0.4 osy to 0.8 osy (about 13.5-27.2 g/m²).

The shaping layer 36 can provide a desired, absorbent retention or storage function, and may include natural fibers (e.g. cellulose fibers), synthetic fibers, superabsorbent materials, a woven fabric a nonwoven fabric; a wet-laid fibrous web; a substantially unbonded airlaid fibrous web; an operatively bonded, stabilized-airlaid fibrous web; a multifunctional stabilized-airlaid fibrous web; or the like, as well as combinations thereof.

In desired configurations, the absorbent body 30 can provide an overall, total absorbent capacity which is at least a minimum of about 10 grams of menses simulant. The overall absorbent capacity can alternatively be at least about 20 grams of menses simulant, and can optionally be at least about 30 grams of menses simulant to provide improved performance. In other aspects, the overall absorbent capacity can be up to a maximum of about 120 grams of menses simulant, or more. The overall absorbent capacity can alternatively be up to about 100 grams of menses simulant, and can optionally be up to about 90 grams of menses simulant to provide improved effectiveness.

In a representative example, the article can include a nonwoven spunbond cover having a basis weight of about 20.3 g/m², and treated with 0.3% AHCOVEL surfactant. The AHCOVEL surfactant acts as a wetting agent to promote liquid intake. The intake layer of the article can comprise a generally homogeneous, stabilized-airlaid fibrous web having a basis weight of about 250 g/m², and a density of about 0.14 g/cm³. The transfer layer can include a rose colored 27.1 g/m² spunbond nonwoven fabric treated with 0.3% AHCOVEL surfactant. The transfer layer can help to regulate liquid transfer from the intake layer to the shaping layer. The shaping layer can be a generally homogeneous, stabilized-airlaid fibrous web having a density of about 0.08 g/cm³ and a basis weight of about 175 g/m². The baffle can be a 0.7 mil (0.018 mm), micro-embossed, polypropylene film, which can substantially prevent liquid from reaching the undergarment and can act as the substrate for the absorbent structure.

The article 20 can include a system of side-panel or wing portions 42. The side-panels can be integrally formed from another component of the article, such as the cover and/or the baffle, and are operatively connected to appointed sections of the side regions 60 along the intermediate portion of the article. Alternatively, the side-panels or wings can be separately provided members that are subsequently attached or otherwise joined to the intermediate portion of the article 20.

The side-panels can have an appointed storage position (e.g. FIGS. 1 and 1A) in which the side-panels 42 are directed generally inwardly toward the longitudinally-extending centerline 52. As illustrated, the side-panel that is connected to one side margin may have sufficient cross-directional length to extend and continue past the centerline 52 to approach the laterally opposite side margin of the article. The storage position of the side-panels can ordinarily represent an arrangement observed when article is first removed from its wrapper or other packaging. Prior to placing the article into a bodyside of an undergarment prior to use, the side-panels 42 can be selectively arranged to extend laterally from the side regions 60 of the article intermediate portion (e.g. FIGS. 2 and 2A). After placing the article in the undergarment, the side-panels 42 can be operatively wrapped and secured around the side edges of the undergarment to help hold the article in place.

Additionally, a selected configuration of garment adhesive 38, such as the illustrated strip regions, may distributed onto the garment-side of the article to help secure the article to the undergarment. Typically, the garment adhesive can be distributed over the garment-side of the baffle, and one or more layers or sheets of release material 40 can be removably placed over the garment adhesive during storage prior to use.

The side-panel portions 42 can have any operative construction, and can include a layer of any operative material. Additionally, each side-panel can comprise a composite material. For example, the side-panels may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web or the like, as well as combinations thereof.

Each side-panel 42 can be joined to its corresponding side region 60 of the article in any operative manner. For example, the side-panel can be joined to the cover 26, the baffle 28 or another article component, as well as any combination thereof. In the illustrated example, each side-panel 42 is joined to the outward, garment-side surface of the baffle 28, but may optionally be joined to the bodyside surface of the baffle. The side-panel can be attached with hotmelt adhesive, but any other operative adhesive or attachment mechanism may alternatively be employed.

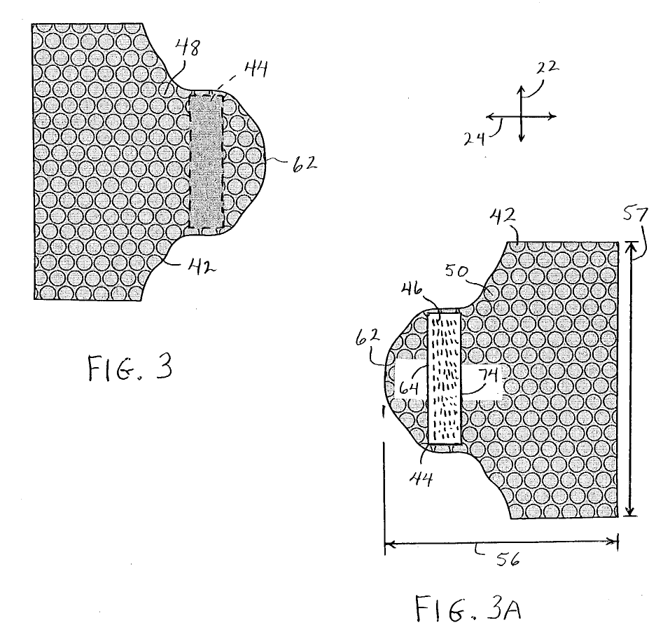

In another feature, each side-panel portion 42, or any desired combination of the employed side-panel portions, can include a panel-fastener component which is operatively joined to an appointed engagement surface of the associated side-panel. With reference to FIGS. 3 and 3A, for example, each side-panel 42 can include a hook or other “male” component 46 of an interengaging mechanical fastener system. Any operative hook component may be employed. For example, a suitable hook component materials can include a J-hook, mushroom-head hook, flat-top nail-head hook, a palm-tree hook, a multiple-J hook or the like, as well as combinations thereof.

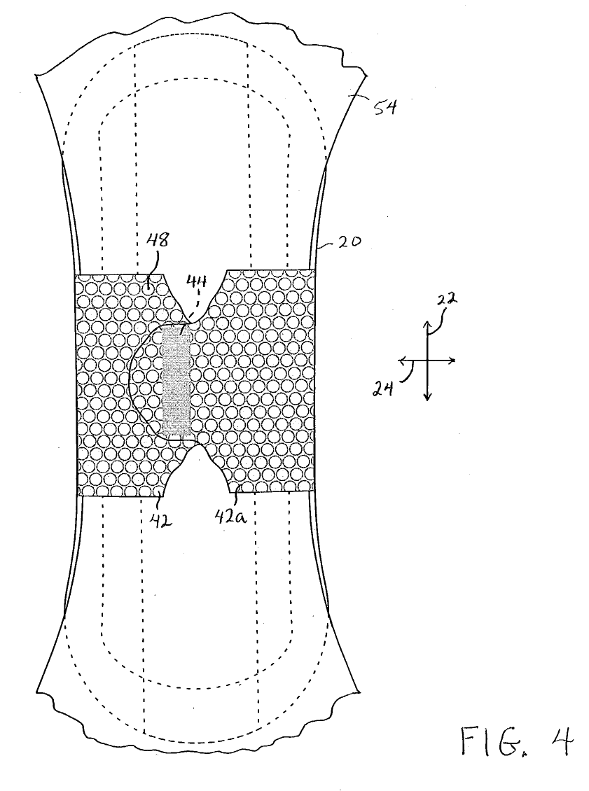

An operative first section of the selected hook component 46 can be joined to a major facing surface of at least a first side-panel portion 42, and can be configured to contact or otherwise engage a second side-panel portion 42a during ordinary use, as representatively shown in FIG. 4. Additionally, an operative second section of a hook component 46a, composed of the same or different type of hook material, can be joined to a major facing surface of the second side-panel portion 42a, and can be configured to contact or otherwise engage an outward surface of the wearer's undergarment 54 during ordinary use. For example, the hook component can be arranged to operatively engage and removably attach to the outward surface of a crotch region of the undergarment.

Each side-panel portion 42, or any desired combination of the employed side-panel portions, can include a loop or other “female” component 48 of an interengaging mechanical fastener system. Any operative loop component may be employed. For example, a suitable loop component material can include a woven fabric, a knit fabric, a nonwoven fabric, a fabric laminated to a substrate or the like, as well as combinations thereof.

An operative first section of a selected loop component 48 can be joined to a major facing surface of at least the second side-panel portion 42a, and can be configured to contact or otherwise engage the hook component 46 on the first side-panel portion 42 during ordinary use, as representatively shown in FIGS. 1A and 4. Additionally, an operative second section of a loop component 48a, composed of the same or different type of loop material, can be joined to a major facing surface of the first side-panel portion 42. As a result, the user can have the option of alternatively attaching the second hook component 46a of the second side-panel onto the second loop component 48a of the first side-panel. Accordingly, the first hook component 46 may alternatively be engaged with the outward surface of the wearer's undergarment.

Each or any desired combination of the provided loop components (48, 48a) may be a separately provided member that is subsequently joined and assembled to its corresponding side-panel portion (42a, 42). In a desired feature, each or any desired combination of the provided loop components can be integrally provided by the material employed to construct its corresponding side-panel portion.

In the various arrangements of the present invention, the hook component 46 can be configured to have a particularly selected hook concentration or density (hooks per unit area). In a particular aspect, the hook density can be at least a minimum of about 1500 hooks/in² (about 232 hooks/cm²). The hook density can alternatively be at least about 2000 hooks/in² (about 310 hooks/cm²), and can optionally be at least about 3000 hooks/in² (about 465 hooks/cm²) to provide improved performance. In another aspect, the hook density can be not more than a maximum of about 7000 hooks/in² (about 1085 hooks/cm²). The hook density can alternatively be not more than about 6000 hooks/in² (about 930 hooks/cm²), and can optionally be not more than about 5000 hooks/in² (about 775 hooks/cm²) to provide improved performance.

If the hook concentration density is outside the desired values, the engagement force between the hook and loop materials can be too low, and can allow the side-panel wings to undesirably unfasten during ordinary use.

Examples of suitable hook materials can include 85-Series and 61-Series hook materials available from Velcro, U.S.A., a business having offices located in Manchester, N.H., U.S.A. The hook materials can, for example, have a hook density of about 775 hooks/cm².

In a particular aspect, the material of the loop component 48 may include a nonwoven fabric having continuous bonded areas defining a plurality of discrete unbonded areas. The fibers or filaments within the discrete unbonded areas of the fabric are dimensionally stabilized by the continuous bonded areas that encircle or surround each unbonded area, such that no support or backing layer of film or adhesive is required. The unbonded areas are specifically designed to afford spaces between fibers or filaments within the unbonded area that remain sufficiently open or large to receive and engage hook elements of the complementary hook material. In particular, a pattern-unbonded nonwoven fabric or web may include a spunbond nonwoven web formed of single component or multi-component melt-spun filaments. At least one surface of the nonwoven fabric can include a plurality of discrete, unbonded areas surrounded or encircled by continuous bonded areas. The continuous bonded areas dimensionally stabilize the fibers or filaments forming the nonwoven web by bonding or fusing together the portions of the fibers or filaments that extend outside of the unbonded areas into the bonded areas, while leaving the fibers or filaments within the unbonded areas substantially free of bonding or fusing. The degree of bonding or fusing within the bonding areas desirably is sufficient to render the nonwoven web non-fibrous within the bonded areas, leaving the fibers or filaments within the unbonded areas to act as “loops” for receiving and engaging hook elements. Examples of suitable point-unbonded fabrics are described in U.S. Patent Application Ser. No. 754,419 entitled PATTERN-UNBONDED NONWOVEN WEB AND PROCESS FOR MAKING THE SAME, by T. J. Stokes et al., and filed Dec. 17, 1996 (attorney docket No. 12,232); the entire disclosure of which is incorporated herein by reference in a manner that is consistent herewith.

Each side-panel portion can be configured to have a distinctive transverse length 56 along the cross-direction 24, and a longitudinal length 57 along the longitudinal direction (e.g. FIG. 3A). In a particular feature, the side-panel transverse length can be at least a minimum of about 50 mm. The transverse length may alternatively be at least about 55 mm, and may optionally be at least about 60 mm to provide improved performance. In another aspect, the side-panel transverse length can be not more than a maximum of about 85 mm. The transverse length may alternatively be not more than about 70 mm, and may optionally be not more than about 65 mm to provide improved performance.

If the transverse length of the side-panels is outside the desired values, the side-panels may be too difficult to attach to each other, may be susceptible to excessive pop-opens, or may leave an excessive amount of overhanging material.

In another feature, the side-panel portions can be configured to provide a selected overlap distance 58 (e.g. FIGS. 1 and 1A). In particular aspects, the side-panel overlap distance can be at least a minimum of about 40 mm. The side-panel overlap distance can alternatively be at least about 42 mm, and can optionally be at least about 45 mm to provide improved performance. In other aspects, the side-panel overlap distance can be up to a maximum of about 60 mm, or more. The side-panel overlap distance can alternatively be up to about 55 mm, and can optionally be up to about 50 mm to provide improved effectiveness.

A further feature of the article can include first and second side-panels which are configured to provide a selected overlap percentage. In a particular aspect, the overlap percentage can be at least a minimum of about 65% when the side panels are arranged or otherwise observed in their storage position. The overlap percentage can alternatively be at least about 70%, and optionally, can be at least about 75% to provide improved benefits. In a further aspect, the overlap percentage can be up to about 100% when the side panels are arranged or otherwise observed in their storage position. The overlap percentage can alternatively be up to about 90% and can optionally be up to about 85% to provide desired benefits.

To determine the overlap percentage, one can employ the following calculation:

overlap percentage=100*(A/B)

where A=side-panel overlap distance 58; and

B=cross-directional distance 59 between the joinder mechanisms that operatively connect the side-panels to the article (e.g. FIG. 1A).

It has been found that the transverse length of the side-panels or wings 42, and the amount of overlap of the side-panels or wings can advantageously improve the ease of fastening and improve the securement of fastening system. If the overlap distance or overlap percentage of the side-panels is outside the desired values, it can be excessively difficult to fasten the side-panels together, or the fastening of the side-panels can be susceptible to excessive pop-opens.

The side panels or wings 42 can be configured such that at least one relatively adjustable fastener component (e.g. a relatively adjustable and repositionable hook component 46) is arranged to fasten and refasten to its cooperating landing-zone component (e.g. the component of loop material 48) at an appointed securement location. In a further feature, each of the first and second side-panels can have a terminal, laterally-distal, panel-edge portion 62, and each panel-edge portion can be laterally spaced from the longitudinally-extending centerline 52 of the article by a panel-edge distance 66 when the side panels are arranged in their storage position. In particular aspects, the panel-edge distance can be at least a minimum of about 5 mm. The panel-edge distance can alternatively be at least about 10 mm, and can optionally be at least about 15 mm to provide improved performance. In other aspects, the panel-edge distance 66 can be up to a maximum of about 32 mm or more. The panel-edge distance can alternatively be up to about 27 mm, and can optionally be up to about 22 mm to provide improved effectiveness. A desired configuration can include a panel-edge distance of about 20 mm. If the panel-edge distance is outside the desired values, the side panels 42 can be less able to resist undesired pop-opens.

In still another feature of the article, each panel-fastener component 44 can have a terminal, laterally-distal, fastener-edge portion 64, and each fastener-edge portion can be laterally spaced from a longitudinally-extending centerline 52 of the article by a selected, outboard fastener-edge distance 68 when the side panels 42 are arranged in their storage position. In particular aspects, the fastener-edge distance 68 can be at least a minimum of about 4 mm. The fastener-edge distance can alternatively be at least about 9 mm, and can optionally be at least about 14 mm to provide improved performance. In other aspects, the fastener-edge distance 68 can be up to a maximum of about 32 mm, or more. The fastener-edge distance can alternatively be up to about 27 mm, and can optionally be up to about 22 mm to provide improved effectiveness. If the fastener-edge distance is outside the desired values, the side panels 42 can be less able to resist undesired pop-opens.

A further feature of the article can be configured with each panel-fastener component having a selected, terminal, inboard-edge portion 74. The inboard-edge portion can be laterally spaced from the longitudinally-extending centerline 52 of the article by an inboard-edge distance 70 when the side panels are arranged in their storage position. In particular aspects, the inboard-edge distance can be at least a minimum of about 1 mm. The inboard-edge distance can alternatively be at least about 3 mm, and can optionally be at least about 6 mm to provide improved performance. In other aspects, the inboard-edge distance 70 can be up to a maximum of about 21 mm, or more. The inboard-edge distance can alternatively be up to about 16 mm, and can optionally be up to about 11 mm to provide improved effectiveness.

While not intending to be bound by any particular theory, it is believed that the resulting, relatively close proximity of the laterally-terminal, end edge of the side-panel to the associated side margin of the article can allow the inner thigh of the wearer to more readily press against the edge of the side-panel and hold the side-panel closed. As a result, the movements of the wearer are less likely to lift the distal edges of the side-panels, and are less likely to prematurely disengage the fastener components. It is also believed that the relatively close proximity of the fastener engagement location to the associated side margin of the article can more readily allow the inner thigh of the wearer to press against the engaged fastener components and hold the fastener system closed. Additionally, the other inner thigh of the wearer can press against the second, relatively adjustable fastener component (e.g. the second hook component 46a) against the outer surface of the undergarment 54. As a result, the second hook component 46a can engage a contacting fabric of the undergarment to further help secure the article 20 to the undergarment 54.

The complementary components of the mechanical fastener are configured to provide a selected attachment peel-force value. In a particular aspect, the peel-force value can be at least a minimum of about 75 grams (g). The peel-force value can alternatively be at least about 100 g, and can optionally be at least about 150 g to provide improved performance. In other aspects, the peel-force value can be up to a maximum of about 300 g, or more. The peel-force value can alternatively be up to about 250 g, and can optionally be up to about 225 g to provide improved effectiveness.

The complementary components of the mechanical fastener are also configured to provide a selected attachment shear-force value. In a particular aspect, the shear-force value can be at least a minimum of about 1000 g. The shear-force value can alternatively be at least about 1250 g, and can optionally be at least about 1500 g to provide improved performance. In other aspects, the shear-force value can be up to a maximum of about 3500 g, or more. The shear-force value can alternatively be up to about 3000 g, and can optionally be up to about 2000 g to provide improved effectiveness.

If the peel-force and/or the shear-force are outside the desired values, the fasteners may experience premature unfastening, or may be too difficult to unfasten to remove the article 20 from an associated undergarment. The peel-force and shear-force values can be determined by employing conventional equipment and procedures.

In the construction of the article 20, the various components may be assembled and held together with any operative securement mechanism or system. For example, the desired attachments or securements can include adhesive bonds, cohesive bonds, thermal bonds, ultrasonic bonds, pins, snaps, staples, rivets, stitches, welds, zippers, or the like, as well as combinations thereof.

The following Examples describe particular configurations of the invention, and are presented to provide a more detailed understanding of the invention. The Examples are not intended to limit the scope of the present invention in any way. From a complete consideration of the entire disclosure, other arrangements within the scope of the claims will be readily apparent to one skilled in the art.

A consumer use study included 200 participants Each participant tested only one code, and was asked to wear a total of eight products. Participants tested two products during daytime hours for a maximum of four days. The participants used the products summarized in the following Table 1.

TABLE 1

| | Side-panel | | | | | |
|---|---|---|---|---|---|---|
| | (wing) | Side-panel | | | | |
| | overlap | edge | Outboard | Inboard | Side-panel | Fastener |
| Product | distance | distance | fastener-edge | fastener-edge | transverse | hook |
| Code | (mm) | (mm) | distance (mm) | distance (mm) | length (mm) | material |
| 1 --G | 40 | 20 | 9 | 1 | 55 | HTH 851 |
| 2 --H | 40 | 20 | 9 | 1 | 60 | HTH 851 |
| 3 --L | 30 | 15 | 4 | 6 | 60 | 85-1001 |
| 4 --N | 44 | 22 | 11 | 1 | 65 | 85-1001 |
| 5 | 30 | 15 | 4 | 6 | | |
| 6 | 30 | 15 | 4 | 6 | | |
| 7 | 30 | 15 | 4 | 6 | | |

The HTH 851 hook material and the 85-1001 hook material were obtained from Velcro USA Inc., Manchester, New Hampshire, U.S.A.
Product Code 5 = Commercially available “Maxi” feminine care, absorbent pad article.
Product Code 6 = Commercially available “Long Super” feminine care, absorbent pad article.
Product Code 7 = Commercially available “Overnight” feminine care, absorbent pad article.

The results of the study indicated that the participants using products with wider side-panel wings having 60 mm and 65 mm of transverse length 56 (e.g. FIG. 3A) (a) experienced fewer problems fastening wings, (b) recorded fewest occurrences of wings becoming unfastened, and (c) considered wing width to be “just right”. The study results also indicated participants using products with the narrowest width wings (55 mm of transverse length), believed wing width was “too short”. The 60 mm wing width appeared to be satisfactory and appeared to be an improvement over the 55 mm wing width. As the wing width is increased (60 mm or more), the satisfaction levels appeared to improve further. The participants appear to have noted a correlation between the wing width and ease of fastening the side-panel wings (longer wing width codes resulted in fewer problems fastening wings.)

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. An absorbent feminine care article having a longitudinal direction, a lateral direction, first and second longitudinally opposed end portions, and a intermediate portion located between said end portions, said article comprising:
a cover;
a baffle;
an absorbent body sandwiched between the cover and baffle;
a first side-panel attached to a first, lateral side region of the intermediate portion of the article, said first side-panel including a first panel-fastener component operatively joined to an engagement surface of said first side-panel; and
a second side-panel attached to a second, lateral side region of the intermediate portion of the article, said second side-panel including a second panel-fastener component operatively joined to an engagement surface of said second side-panel; wherein
the first and second side panels have an overlap distance of at least a minimum of about 40 mm when the side panels are arranged in their storage position.

2. An article as recited in claim 1, wherein said absorbent body includes a pad shaping layer which is positioned between said cover and said baffle.

3. An article as recited in claim 2, wherein said absorbent body includes an intake layer which is positioned between said cover and said pad shaping layer.

4. An article as recited in claim 3, wherein said absorbent body includes a transfer layer which is positioned between said intake layer and said pad shaping layer.

5. An article as recited in claim 2, wherein said pad shaping layer has a total absorbent capacity of at least about 50 grams of menses simulant.

6. An article as recited in claim 1, wherein each said first and second side-panels has a terminal, laterally-distal, panel-edge portion; and each panel-edge portion is laterally spaced from a longitudinally-extending centerline of the article by a panel-edge distance which is at least a minimum of about 10 mm when the side panels are arranged in their storage position.

7. An article as recited in claim 1, wherein each said panel-fastener component has a terminal, laterally-distal, fastener-edge portion; and each fastener-edge portion is laterally spaced from a longitudinally-extending centerline of the article by a fastener-edge distance which is at least a minimum of about 4 mm when the side panels are arranged in their storage position.

8. An article as recited in claim 1, wherein said first and second side-panels have an overlap distance which is at least a minimum of about 42 mm when the side panels are arranged in their storage position.

9. An article as recited in claim 1, wherein said first and second side-panels have an overlap percentage which is at least a minimum of about 60% when the side panels are arranged in their storage position.

10. An article as recited in claim 1, wherein each panel-fastener component has a terminal, inboard-edge portion; and each inboard-edge portion is laterally spaced from a longitudinally-extending centerline of the article by an inboard-edge distance which is at least a minimum of about 1 mm when the side panels are arranged in their storage position.

11. An article as recited in claim 1, wherein each panel-fastener component has a hook concentration of at least about 232 hooks/cm².

12. An article as recited in claim 11, wherein each panel-fastener component has a hook concentration of at least about 465 hooks/cm².

13. An article as recited in claim 11, wherein each panel-fastener component has a hook concentration of not more than about 1085 hooks/cm².

14. An article as recited in claim 1, wherein each panel-fastener component has a hook concentration of at least 465 hooks/cm².

15. An article as recited in claim 11, wherein each panel-fastener component has a hook concentration of not more than about 1085 hooks/cm².

16. An article as recited in claim 1, wherein
each said first and second side-panels has a terminal, laterally-distal, panel-edge portion; and each panel-edge portion is laterally spaced from a longitudinally-extending centerline of the article by a panel-edge distance which is at least a minimum of about 10 mm when the side panels are arranged in their storage position;
each said panel-fastener component has a terminal, laterally-distal, fastener-edge portion; and each fastener-edge portion is laterally spaced from a longitudinally-extending centerline of the article by a fastener-edge distance which is at least a minimum of about 4 mm when the side panels are arranged in their storage position;
each panel-fastener component has a hook concentration of at least about 232 hooks/cm²

17. An article as recited in claim 1, wherein
each said first and second side-panels has a terminal, laterally-distal, panel-edge portion;
and each panel-edge portion is laterally spaced from a longitudinally-extending centerline of the article by a panel-edge distance which is at least a minimum of about 10 mm when the side panels are arranged in their storage position;
each said panel-fastener component has a terminal, laterally-distal, fastener-edge portion; and each fastener-edge portion is laterally spaced from a longitudinally-extending centerline of the article by a fastener-edge distance which is at least a minimum of about 9 mm when the side panels are arranged in their storage position;
each panel-fastener component has a hook concentration of at least about 310 hooks/cm².

18. An article as recited in claim 1, wherein
each said first and second side-panels has a terminal, laterally-distal, panel-edge portion; and each panel-edge portion is laterally spaced from a longitudinally-extending centerline of the article by a panel-edge distance which is at least a minimum of about 15 mm when the side panels are arranged in their storage position;
each said panel-fastener component has a terminal, laterally-distal, fastener-edge portion; and each fastener-edge portion is laterally spaced from a longitudinally-extending centerline of the article by a fastener-edge distance which is at least a minimum of about 9 mm when the side panels are arranged in their storage position;
each panel-fastener component has a hook concentration of at least about 310 hooks/cm².

19. An article as recited in claim 1, wherein
the first and second side panels have an overlap distance of at least a minimum of about 42 mm when the side panels are arranged in their storage position;
each said first and second side-panels has a terminal, laterally-distal, panel-edge portion; and each panel-edge portion is laterally spaced from a longitudinally-extending centerline of the article by a panel-edge distance which is at least a minimum of about 15 mm when the side panels are arranged in their storage position;
each said panel-fastener component has a terminal, laterally-distal, fastener-edge portion; and each fastener-edge portion is laterally spaced from a longitudinally-extending centerline of the article by a fastener-edge distance which is at least a minimum of about 4 mm when the side panels are arranged in their storage position;
each panel-fastener component has a hook concentration of at least about 465 hooks/cm².

20. An article as recited in claim 1, wherein
the first and second side panels have an overlap distance of at least a minimum of about 45 mm when the side panels are arranged in their storage position;
each said first and second side-panels has a terminal, laterally-distal, panel-edge portion;
and each panel-edge portion is laterally spaced from a longitudinally-extending centerline of the article by a panel-edge distance which is at least a minimum of about 15 mm when the side panels are arranged in their storage position;
each said panel-fastener component has a terminal, laterally-distal, fastener-edge portion; and each fastener-edge portion is laterally spaced from a longitudinally-extending centerline of the article by a fastener-edge distance which is at least a minimum of about 9 mm when the side panels are arranged in their storage position;
each panel-fastener component has a hook concentration of at least about 465 hooks/cm².
